(19) United States
(12) Patent Application Publication
(10) Pub. No.: US 2009/0169651 A1
(43) Pub. Date: Jul. 2, 2009

(54) ANTI-SKIN DAMAGE COMPOSITIONS WITH COMPLIMENTARY DUAL MODE OF ACTION

(76) Inventor: Muhammed Majeed, Piscataway, NJ (US)

Correspondence Address: SABINSA CORPORATION, 70 ETHEL ROAD WEST, UNIT 6, PISCATAWAY, NJ, 08854, US

(21) Appl. No.: 12/164,052

(22) Filed: Jun. 28, 2008

Related U.S. Application Data
(60) Provisional application No. 60/946,740, filed on Jun. 28, 2007.

(51) Int. Cl. A61K 36/889; A61P 17/00

(52) U.S. Cl. 424/727

(57) ABSTRACT

Disclosed are skin care compositions exerting a novel complementary dual mode of action in protecting the skin from day-to-day insults. The skin care compositions of the present invention comprise the extract from the liquid endosperm of Cocos nucifera as the principle ingredient along with one or more actives including the fruit rind extracts of Garcinia cambogia, Garcinia indica and Garcinia mangostana, seed coat extract of Tamarindus indicus, Chlorogenic acid extract from the beans of Coffea arabica, seed extracts of Nelumbo species, the leaf extracts of Murraya koenigii and the triterpene pentapeptides of oleanolic acid and ursolic acid. While the extract of Cocos nucifera has been included as a nutritional supplement (a natural cell culture medium) to ensure the maintenance of the viability and constant renewal of the cells of the skin, the other actives have been carefully chosen based on one or more of their effective skin care properties. The compositions of the present invention offer a safe prophylactic/therapeutic management solution to the wide range of pathological states associated with skin insults and the associated psycho-social issues.

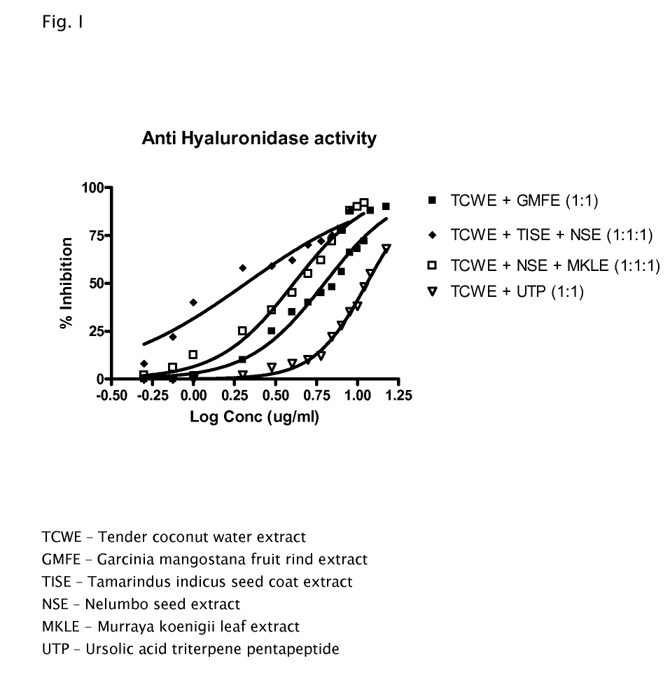

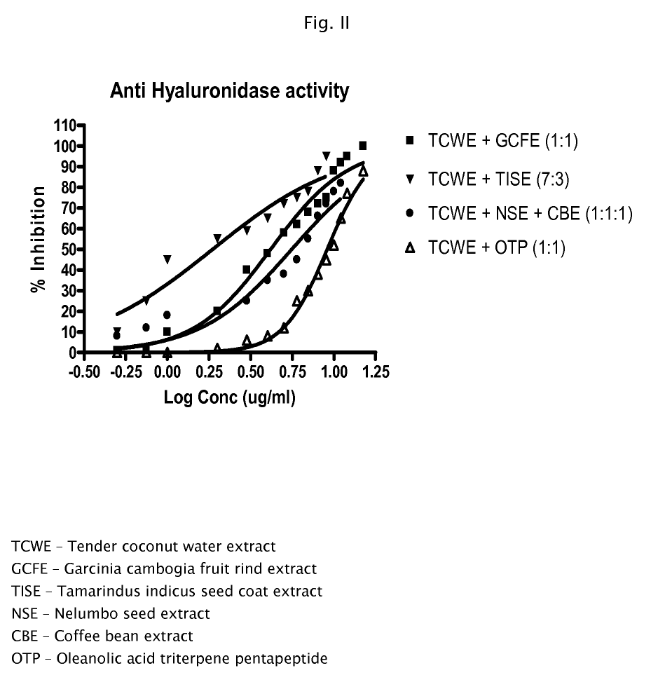

This application is a non-provisional filing of provisional application No. 60/946,740 filed on Jun. 28, 2007, the contents of which are incorporated herewith by reference.

The invention generally relates to personal care compositions. In particular, the invention relates to skin care compositions made from natural plant extracts.

### BACKGROUND OF THE INVENTION

The skin is the largest organ of the human body. The skin is primarily responsible for:
a) Protecting the internal organs of the body.
b) Protecting against heat, cold, and physical injuries.
c) Providing sensory information about the nature of the external world
d) Providing a first line of defense against invasion by bacteria, viruses, and other toxic elements
e) Functioning as an excretory organ, removing toxins from the body via perspiration.

Skin insult and subsequently aging occur as a consequence of accumulated cellular damage. A series of both extrinsic and intrinsic factors contribute to skin damage. The extrinsic factors include oxidative processes and related free radical (nitric oxide, superoxide radicals, hydrogen peroxide, and hydroxide free radicals) damage that result from UV sunlight, smog, environmental toxins, cigarette smoke, poor diet, excess alcohol consumption, stress, harsh soaps or detergent-based moisturizers and sleep deprivation. The intrinsic factors include genetically predetermined cell cessation, chronological aging, hormone depletion and immune suppression.

Young skin is also exposed to these potentially damaging changes. But, when young, there is sufficient cellular energy (ATP) for DNA repair and cell renewal. Enzymes that provide antioxidant activity such as SOD and catalase are readily available. As skin ages, there is increased wear and tear, while at the same time the energy for cell repair and renewal is diminished and the antioxidant enzymes are less available.

Typical skin damage includes thinning of the skin, reduction in the cells and blood vessels that supply the skin as well as a flattening of the dermal-epidermal junction resulting in weaker mechanical resistance of this junction, appearance of fine lines and wrinkles, hyperpigmentation, sallowness, sagging, dark under-eye circles, puffy eyes, enlarged pores, diminished rate of cell turnover, and abnormal desquamation or exfoliation. Additional damage may include visible dead skin caused due to flaking and scaling.

Skin damage must be tackled by two complementary mechanisms. (a) A continued cell repair and renewal; (b) A protective mechanism that would effectively tackle various forms of extrinsic skin damage including oxidative processes and related free radical (nitric oxide, superoxide radicals, hydrogen peroxide, and hydroxide free radicals) damage that result from UV sunlight, smog, environmental toxins, cigarette smoke, poor diet, excess alcohol consumption, stress, harsh soaps or detergent-based moisturizers and sleep deprivation. Herbal treatments for the prevention of the skin damage having gained momentum in recent years in view of their enhanced safety profile. Carefully chosen herbs known for their efficacy in ameliorating the deleterious effects on skin based on the study of their biological properties have been included in the present invention. The descriptions herein below seek to elucidate important properties of the herbal components included in the present invention in relation to the available prior art.

Extract from the Liquid Endosperm of Cocos nucifera (Tender Coconuts)

The extract occurs as an off white to cream powder with a mild characteristic odor. The extract is hygroscopic. The active components of the extract include amino acids, carbohydrates, essential minerals and enzymes. The extract nurtures and serves as a natural reservoir of nutrients to promote tissue growth. The following properties of tender coconut water have been documented by the Coconut Development Board of India-Food and Agriculture Organization of the United Nations.

Coconut water,
a) Contains organic compounds possessing growth promoting properties.
b) Aids in keeping the skin from itching, application on the body also helps prevent prickly heat, and summer boils, and helps to subside the effects of rashes caused by chicken pox, hives, measles, small pox, mosquito and insect bites.
c) Help prevent bacterial, viral, and fungal infections.
d) Helps eliminate Candida yeast infections.
e) Helps eradicate eczema.
f) Helps keep skin soft and smooth.
g) Helps prevent dandruff.
h) Helps prevent premature aging and wrinkling of the skin.
i) Helps prevent skin cancer and other blemishes.
j) Helps prevent dry and chapped skin.

Fruit Rind Extract from Garcinia cambogia and Garcinia indica

The fruit rind extract from Garcinia Cambogia and Garcinia indica occurs as a pale brownish yellow powder with a characteristic odor. The extract shows stability at 40° C./RH 75% for 2 months. Therefore the product may be stable for at least 2 years under ambient condition. The active components of the extract include phenolic xanthones with purity not less than 95%. The product is characterized by (a) Anti-oxidant activity (b) Anti-inflammatory activity (c) Cyclooxygenase inhibitory activity (d) UV A and UV B protective activity and (e) Hyper proliferation inhibitory activity. The extract may be combined with other moisturizers like coriander seed oil, avocado oil, sandal seed oil, xyloglucans, beta-glycans and the like. Synergistic combinations with other known elastase inhibitors, collagenase inhibitors, and anti-phlogistic, and anti-oxidant actives are also proposed. The antioxidant activity of the fruit rind extract of Garcinia cambogia and Garcinia indica has been reported as prior art. The extract has been reported protective at concentrations between 30-90 micromol/L by decreasing H₂O₂-induced DNA strand breaks and oxidized bases in Cell Biol Toxicol. 2004 March; 20(2):71-82. The antioxidant activity of Garcinia indica (kokam) and its syrup has also been reported in Current Science, Vol. 91, No. 110 Jul. 2006. The anti-inflammatory ability of the extract evinced by the suppression of inducible nitric oxide synthase and cyclooxygenase-2 in down-regulating nuclear factor-kappa B pathway has been reported in Mol. Carcinog. 2004 November; 41(3) 140-9.

Extract of Garcinia mangostana

The fruit rind extract of Garcinia Mangostana occurs as a yellow powder with mild characteristic odor. The extract shows stability at 40° C./RH 75% for 2 months. The extract may therefore be stable for at least 2 years under ambient conditions. The active component of the extract is α-Mangostin with purity not less than 48%. The extract exhibits (a) Anti-oxidant activity as reported in the publication titled “Antioxidant xanthones from the pericarp of Garcinia mangostana (Mangosteen)” authored by Jung H A, Su B N, Keller W J, Mehta R G, Kinghorn A D, published in J Agric Food Chem. 2006 Mar. 22; 54(6):2077-82 and in the Japanese Patent No. JP8225783 to Yoshikawa Masayuki; Yoshizumi Tomoji titled “Natural antioxidant containing gamma-mangostin as active ingredient” published on Sep. 3, 1996. (b) Anti-inflammatory activity as reported in the publication titled “Inhibition of cyclooxygenase and prostaglandin E2 synthesis by alpha.-mangostin, a xanthone derivative in mangosteen, in C6 rat glioma cells” authored by Nakatani K, Nakahata N, Arakawa T, Yasuda H, Ohizumi Y and published in Biochem Pharmacol. 2002 Jan. 1; 63(1):73-9. (c) activity (Anti-elastase/MMP inhibitory activities) as reported in U.S. Publication No. 20040077523 titled “Method for pressuring reduction of elasticity of skin” to Ochiai Nobuhiko; Inomata Shinji; Takada Keiko on Apr. 22, 2004. (d) Anti-acne activity as reported in the Japanese Patent No. 9095453 titled “Antimicrobial composition” to Yamaguchi Norio; Yuasa Katsumi on Apr. 8, 1997 and in the Japanese Publication No. 2000229857 titled “Testosterone-5-alpha-reductase inhibitor” to Kubo Michitoku; Matsuda Hideaki; linuma Munekazu; Tosa Hideki on Aug. 22, 2000 (e) Anti-tumor activity as reported in Japanese Patent No. 10203977 titled “Antitumor Agent” to linuma Munekazu; Nozaki Hiroshi on Aug. 4, 1998 (f) UV A and UV B protective activity as reported in the French Patent No. 2754447 titled “Sun-cream preparation” to Gedouin Jean; Vallee Romuald on Apr. 17, 1998 and in the Japanese Patent No. 4244004 titled “Cosmetic” to Monobe Akio; Kawaguchi Shigetaka; Katada Tomonori on Sep. 1, 1992.

Indian Date Extract (Tamarind Seed Coat Extract)

The extract exhibits a Pinkish brown color with characteristic odor. The extract shows stability at 40° C./RH 75% for 2 months. The extract may therefore be stable for at least 2 years under ambient conditions. The active ingredients of the extract are polyphenols. The extract exhibits (a) Antioxidant Activity reported in the publication titled “Antioxidative components isolated from the seed of tamarind (Tamarindus indica L.)” published in J. Agric. Food chem. 1994, 42, 2671-2674 (b) activity reported in Japanese Patent No. 8231347 titled “Hyaluronidase inhibitor and cosmetic” to Kanbara Toshimitsu; Kanechika Rikako; Tamura Kokichi on Sep. 10, 1996.(c) Tyrosinase inhibitory activity reported in Japanese Patent No. 8231343 titled “Tyrosinase inhibitor, whitening cosmetic and discoloration-preventing agent” to Tamura kokichi; Oda mayumi; Imoto tomoko on Sep. 10, 1996.

Green Coffee Bean Extract—Chlorogenic Acid

Chlorogenic extracts from green coffee beans has been discussed in several prior art references. Chlorogenic acid as an anti-oxidant and anti-peroxidant has been discussed in Duke, James A. 1992. Handbook of biologically active phytochemicals and their activities. Boca Raton, Fla. Chlorogenic acid as a lipoxygenase-inhibitor has been discussed in Oszmianski, J. and Lee, C. Y. 1990. Inhibitory Effect of Phenolics on Carotene Bleaching in Vegetables. J. Agric. Food Chem. 38: 688-690. Chlorogenic acid as aldose-reductase inhibitor has been discussed in Yoshikawa, M., et al. 1999. Medicinal Flowers. 1. Aldose Reductase Inhibitors and Three New Eudesmane-Type Sesquiterpenes, Kikkanols A, B, and C, From the Flowers of Chrysanthemum inducum L. Chem Pharm Bull, 47(3): 340-345. The anti-inflammatory activity of chlorogenic acid as a leukotriene inhibitor and histamine inhibitor, the anti-tumor promotion and anti-UV activities have been documented in Duke, James A. 1992. Handbook of biologically active phytochemicals and their activities. Boca Raton, Fla.

Seed Extracts from Lotus (Nelumbo Species)

Methyl transferase enzymes and other constituents of the Lotus seeds are believed to prevent the accumulation of damaged proteins within these seeds and thus are considered to possess significant activity.

Compositions for the treatment of the skin that contain an extract from plants of the genus Nelumbo are reported in U.S. Pat. No. 6,468,564 titled “Topical compositions containing lotus for skin treatment” to Riley, Patricia A. and Babcock, Thomas on Oct. 22, 2002.

U.S. Pat. No. 5,925,348 titled “Methods utilizing compositions containing sacred lotus (methyltransferase) to treat aging skin” to Riley, Patricia A. Babcock, Thomas on Jul. 20, 1999 also discusses Cosmetic, dermatological and dietary compositions for treatment of aging of the skin are provided that contain an extract of the Sacred Lotus (Nelumbo Nucifera) seed as an active agent in a suitable carrier or vehicle along with methods of treatment to reduce signs of aging such loss of elasticity, age spots, acne, blemishes, enlarged pores, fine lines, wrinkles and to promote overall younger looking skin by using said compositions.

Leaf Extracts from Murraya koenigii

The anti-oxidant properties of the leaves of Murraya koenigii have been documented in a number of research publications, some of which are referred herein below.

The beneficial effects of Murraya koenigii in combating oxidative stress and oxidative damage due to the deleterious effects of free radical generation has been reported in the publication titled “Beneficial effects of Murraya koenigii leaves on antioxidant defense system and ultra structural changes of pancreatic beta-cells in experimental diabetes in rats”, authored by Arulselvan P, Subramanian S P in the Chem Biol Interact. 2007 Jan. 30; 165(2):155-64. Epub 2006 Dec. 2.

A dose-dependent Nitric Oxide scavenging activity of Murraya koenigii has been reported in the publication titled “Evaluation of nitric oxide scavenging activity of certain spices in vitro: a preliminary study”, authored by Baliga M S, Jagetia G C, Rao S K, Babu K. in Nahrung. 2003 August; 47(4):261-4.

The structure-function relationship of carbazoles extracted from Murraya koenigii in terms of their ability to act as natural anti-oxidants to prevent the deleterious effects of lipid peroxidation has been reported in the publication titled “Phenolic antioxidants from herbs and spices”, authored by Nakatani N. in Biofactors. 2000; 13(1-4):141-6.

Dermal Matrix Stimulating Peptides

The significance of topical peptides in stimulating the upper dermis of the skin to renew itself by cellular re-growth has been discussed in several prior art patent references including:

U.S. Pat. No. 6,492,326 titled “Skin care compositions containing combination of skin care actives” to Robinson, Larry Richard; Bissett, Donald Lynn; Deckner, George Endel; Ha, Robert Bao Kim on Dec. 10, 2002.

U.S. Pat. No. 6,620,419 titled “Cosmetic or dermopharmaceutical use of peptides for healing, hydrating and improving skin appearance during natural or induced ageing (heliodermia, pollution)” to Lintner, Karl on Sep. 16, 2003 discloses peptide formulas of the general sequence palmitoyl-lysyl-threonyl-lysyl-serine that increase the synthesis of collagen and gylcosaminoglycans. They act synergistically to heal wrinkles and other forms of skin aging far more effectively than earlier formulations.

The Lintner patent is the basis for the commercial product Matrixyl® (Sederma, SA). By virtue of published research Matrixyl™ is recognized as a cosmetic substance that increases and restores the formative cells, fibro-collagenous network and extra-cellular components. (Lintner K, Preschard, O., “Biologically Active Peptides: From A Laboratory Bench Curiosity to a Functional Skin Care Product”, International Journal of Cosmetic Science, 22, 207-218 (2000)) and (Mas-Chamberlin et al. “Matrikines: A Natural Concept to Replace Retinoids in Wrinkle Treatment, Personal Care Asia Pacific, 5, 1, (2004)). The net result is to increase the thickness of the skin by restoring the reparative capacity of the upper dermis. Consequently, the skin is better able to withstand the deformation imposed on it by the active contraction and relaxation of expression muscles, and micro-contractions within the skin itself. The latest peptide formulations to appear in the art, i.e., the oligopeptides, tetrapeptides and tripeptides are described by Lintner in (US Publication # 20040132667), and are marketed by Sederma, SA. as Matrixyl™ 3000. Matrixyl™ 3000 is defined as a mixture of Glycerin, water, butylene glycol, carbomer, polysorbate and palmitoyl oligopeptide and palmitoyl terapeptide in the INCI classification. According to published clinical data by Sederma, SA. Matrixyl™ 3000 is much faster at reducing wrinkles then the original Matrixyl™ formula. The use of a combination of peptides that regenerate the dermal matrix in a cosmetic application has been discussed in U.S. Publication. No. 20070048245 titled “Cosmetic composition to accelerate repair of functional wrinkles “to Belfer, William A. on Mar. 1, 2007.

It is the principle object of the present invention to develop skin care compositions containing natural plant extracts, wherein the compositions exhibit a complimentary dual mode of action by (a) ensuring the viability and constant renewal of the cells of the skin; (b) ensuring skin protection by virtue of “skin care” properties of other natural actives incorporated in the composition.

It is another object of the present invention to develop skin care compositions containing natural plant extracts, wherein each of the natural extracts have been carefully chosen based on useful cosmetic properties.

It is yet another object of the present invention to develop skin care compositions containing natural plant extracts that exhibits an enhanced safety profile for application to the skin.

It is yet another object of the present invention to develop skin care compositions containing natural plant extracts that exhibits an enhanced ability to counter the problems of both intrinsic and extrinsic skin aging through complimentary effects.

Further, the present invention seeks to develop skin care compositions containing natural plant extracts wherein the compositions are suited to address specific problems or a combination of one or more problems or the multitude of problems associated with skin insult, including but not limited to thinning of the skin, reduction in the number of skin cells, reduction in the number of blood vessels that supply the skin and hence reduction in the microcirculation of the skin, flattening of the dermal-epidermal junction which may cause weaker mechanical resistance of the skin, appearance of fine lines, wrinkles (both the superficial wrinkles and deep/coarse wrinkles), crevices, bumps, hyper-pigmentation in the forms of age spots and freckles, sallowness, sagging, loss of skin firmness, loss of skin elasticity, skin blotching, dark under-eye circles, puffy eyes, enlarged skin pores, diminished rate of cell turnover, abnormal desquamation or exfoliation or the skin cells and visible dead skin caused due to flaking and scaling.

Still further, the present invention seeks to develop skin care compositions containing natural plant extracts wherein the compositions are suited to address specific problems or combination of one or more problems or the multitude of problems associated with skin aging through a combination of various mechanisms including not limited to rejuvenation of skin by providing nutrition, improving the cell turn over, improving skin microcirculation, stimulating collagen and elastin fiber synthesis, inhibition of serine proteases such as collagenases and elastases from acting on collagen and elastin in the dermis of the skin, maintenance of the “acid mantle” produced by the sebaceous and sweat glands to confer a healthy glow to the skin, relaxation of the skin, soothening and softening dry skin and providing powerful anti-oxidant systems that will effectively counter the deleterious effects of free radicals like nitric oxide, superoxide radicals, hydrogen peroxide, and hydroxide free radicals generated by oxidative processes.

The present invention also aims at develop skin care compositions containing natural plant extracts wherein the said composition exhibits enhanced stability by virtue of the stability evinced by the individual plant extracts included therein.

The present invention also aims at develop skin care compositions containing natural plant extracts which can be combined with one or more cosmetic or manufacturing adjuncts including but not limited to sunscreen agents, skin-lightening agents, skin-tanning agents, other antioxidants, perfumes, opacifiers, preservatives, colorants, emulsifiers, thickeners, buffers and the like.

The present invention also aims to develop skin care compositions containing natural plant extracts that can be administered by the topical mode or oral mode or co-administered by both the topical and oral modes.

The present invention also aims to provide a natural, comprehensive, multi-component, multi-functional, safe and stable cosmetic solution for the entire gamut of pathological changes associated with skin insults.

The present invention also aims to provide natural, comprehensive, multi-component, multi-functional, safe and stable cosmetic solution that would help individuals manage the psychosocial aspects of poor skin condition, including but not limited to social anxiety, social isolation, poor self image, eating disorders, body dysmorphic disorder, chronic illnesses, workplace discrimination, lack of confidence and depression.

The present invention fulfills these objectives and provides further related advantages.

### SUMMARY OF THE INVENTION

The present invention relates to anti-skin damage compositions exerting a novel complementary dual mode of action in protecting the skin from day-to-day insults. The skin care compositions of the present invention comprise the extract from the liquid endosperm of Cocos nucifera as the principle ingredient along with one or more actives including the fruit rind extracts of Garcinia cambogia, Garcinia indica and Garcinia mangostana, seed coat extract of Tamarindus indicus, Chlorogenic acid extract from the beans of Coffea arabica, seed extracts of Nelumbo species, the leaf extracts of Murraya koenigii and the triterpene pentapeptides of oleanolic acid and ursolic acid. While the extract of Cocos nucifera has been included as a nutritional supplement (a natural cell culture medium) to ensure the maintenance of the viability and constant renewal of the cells of the skin, the other actives have been carefully chosen based on one or more of their effective skin care properties. The compositions of the present invention offer a safe prophylactic/therapeutic management solution to the wide range of pathological states associated with skin insults and the associated psycho-social issues.

The invention presents the following advantageous features.
a) The present invention provides skin care compositions containing natural plant extracts, wherein the compositions exhibit a dual mode of action by (a) ensuring the viability and constant renewal of the cells of the skin; (b) ensuring skin protection by virtue of “skin care” properties of other natural actives incorporated in the composition.
b) The present invention provides skin care compositions containing natural plant extracts, wherein each of the natural extracts have been carefully chosen based on useful cosmetic properties.
c) The present invention provides skin care compositions containing natural plant extracts that exhibits an enhanced safety profile for application on the skin.
d) The present invention provides skin care compositions containing natural plant extracts that exhibits an enhanced ability to counter the problems of both intrinsic and extrinsic skin insults, through a complimentary effect.
e) The present invention provides skin care compositions containing natural plant extracts wherein the compositions are suited to address the multitude of problems associated with skin insult including but not limited to thinning of the skin, reduction in the number of skin cells, reduction in the number of blood vessels that supply the skin and hence reduction in the microcirculation of the skin, flattening of the dermal-epidermal junction which may cause weaker mechanical resistance of the skin, appearance of fine lines, wrinkles (both the superficial wrinkles and deep/coarse wrinkles), crevices, bumps, hyper-pigmentation in the forms of age spots and freckles, sallowness, sagging, loss of skin firmness, loss of skin elasticity, skin blotching, dark under-eye circles, puffy eyes, enlarged skin pores, diminished rate of cell turnover, abnormal desquamation or exfoliation or the skin cells and visible dead skin caused due to flaking and scaling.
f) The present invention provides skin care compositions containing natural plant extracts wherein the compositions are suited to address specific problems or combination of one or more problems or the multitude of problems associated with skin insult through a combination of various mechanisms including not limited to rejuvenation of skin by providing nutrition, improving the cell turn over, improving skin microcirculation, stimulating collagen and elastin fiber synthesis, inhibition of serine proteases such as collagenases and elastases from acting on collagen and elastin in the dermis of the skin, maintenance of the “acid mantle” produced by the sebaceous and sweat glands to confer a healthy glow to the skin, relaxation of the skin, soothening and softening dry skin and providing powerful anti-oxidant systems that will effectively counter the deleterious effects of free radicals like nitric oxide, superoxide radicals, hydrogen peroxide, and hydroxide free radicals generated by oxidative processes.
g) The present invention provides skin care compositions containing natural plant extracts, wherein the said composition exhibits enhanced stability by virtue of the stability evinced by the individual plant extracts included therein.
h) The present invention provides skin care compositions containing natural plant extracts which can be combined with one or more cosmetic or manufacturing adjuncts including but not limited to sunscreen agents, skin-lightening agents, skin-tanning agents, other antioxidants, perfumes, opacifiers, preservatives, colorants, emulsifiers, thickeners, buffers and the like.
i) The present invention provides skin care compositions containing natural plant extracts which can be administered by the topical mode or oral mode or co-administered by both the topical and oral modes.
j) The present invention provides a natural, comprehensive, multi-component, multi-functional, safe and stable prophylactic and therapeutic solution for the entire gamut of pathological changes associated with skin insults.
k) The present invention also provides a natural, comprehensive, multi-component, multi-functional, safe and stable prophylactic and therapeutic solution that would help individuals manage the psychosocial aspects of skin insults, including but not limited to social anxiety, social isolation, poor self image, eating disorders, body dysmorphic disorder, chronic illnesses, workplace discrimination, lack of confidence and depression.

Other features and advantages of the present invention will become apparent from the following more detailed description, taken in conjunction with the accompanying drawings, which illustrate, by way of example, the principle of the invention.

### BRIEF DESCRIPTION OF THE DIAGRAMS

FIGS. I and II show graphical representations of the synergistic anti-hyaluronidase activities exhibited by the compositions of the present invention discussed herein below.

### DETAILED DESCRIPTION OF THE PREFERRED EMBODIMENT

In the most preferred embodiment, the present invention relates to anti-skin damage compositions, said compositions protecting the skin from insult through a dual complementary mode of action involving (i) continued cell repair and renewal mechanism; and (ii) protection against various forms of extrinsic skin damage. More specifically, in preferred embodiments the present inventions relates to the following anti-skin damage compositions.
1. Anti-skin damage compositions, said compositions comprising the extract of tender coconut water and the fruit ring extract of Garcinia cambogia in a concentration ratio of 1:1. More specifically, the said compositions comprise from about 50 mg to 500 mg of tender coconut water extract. Further, the said compositions comprise from about 50 mg to 500 mg of the fruit ring extract of Garcinia cambogia. While the extract of tender coconut water provides a continued cell repair and renewal mechanism, the fruit ring extract of Garcinia cambogia provides a protective mechanism to effectively tackle various forms of extrinsic skin damage. Still further, the extract of tender coconut water and the fruit ring extract of Garcinia cambogia at a concentration ratio of 1:1 synergistically shows a hyaluronidase activity of IC50 at 4.3 μg/ml.
   2. Anti-skin damage compositions, said compositions comprising the extract of tender coconut water and the fruit ring extract of Garcinia mangostana at a concentration ratio of 1:1. More specifically, the said compositions comprise from about 50 mg to 500 mg of tender coconut water extract. Still more specifically, the said compositions comprise from about 50 mg to 500 mg of the fruit ring extract of Garcinia mangostana. While the extract of tender coconut water provides a continued cell repair and renewal mechanism, the fruit ring extract of Garcinia mangostana provides a protective mechanism to effectively tackle various forms of extrinsic skin damage. Further, the extract of tender coconut water and the fruit ring extract of Garcinia mangostana at a concentration ratio of 1:1 synergistically shows a hyaluronidase activity of IC50 at 7.6 μg/ml.
   3. Anti-skin damage compositions, said compositions comprising the extract of tender coconut water and the seed coat extract of Tamarindus indicus at a concentration ratio of 7:3. More specifically, said compositions comprising from about 70 mg to 700 mg of tender coconut water extract. Further, the said compositions comprise from about 30 mg to 300 mg of the seed coat extract from Tamarindus indicus. While the extract of tender coconut water provides a continued cell repair and renewal mechanism, the seed coat extract of Tamarindus indicus provides a protective mechanism to effectively tackle various forms of extrinsic skin damage. Still further, the extract of tender coconut water and the seed coat extract of Tamarindus indicus at a concentration ratio of 7:3 show synergistic anti-hyaluronidase activity with an IC50 (μg/ml) of 1.2.
   4. Anti-skin damage compositions, said compositions comprising the extract of tender coconut water, the seed coat extract of Tamarindus indicus and the seed extract from the members of the genus Nelumbo at a concentration ratio of 1:1:1. Specifically, the said compositions comprise
   1) from about 100 mg to 1000 mg of tender coconut water extract;
   2) from about 100 mg to 1000 mg of the seed coat extract from Tamarindus indicus; and
   3) from about 100 mg to 1000 mg of seed extract from the members of the genus Nelumbo.
   While the extract of tender coconut water provides a continued cell repair and renewal mechanism, the seed coat extract of Tamarindus indicus and the seed extract from the members of the genus Nelumbo provide a protective mechanism to effectively tackle various forms of extrinsic skin damage. Further, the extract of tender coconut water, the seed coat extract of Tamarindus indicus and the seed extract from the members of the genus Nelumbo at a concentration ratio of 1:1:1 shows synergistic anti-hyaluronidase activity with an IC50 (μg/ml) of 1.58.
5. Anti-skin damage compositions, said compositions comprising:
   1) extract of tender coconut water;
   2) seed extract from the members of the genus Nelumbo; and
   3) Chlorogenic acid extract from the green beans of Coffea Arabica
   at a concentration ratio of 1:1:1.
   Specifically, the said composition comprises:
   1) from about 100 mg to 1000 mg of tender coconut water extract;
   2) from about 100 mg to 1000 mg of seed extract from the members of the genus Nelumbo; and
   3) from about 100 mg to 1000 mg of Chlorogenic acid extract from the green beans of Coffea Arabica.
   More specifically, 1) the extract of tender coconut water; 2) seed extract from the members of the genus Nelumbo; and 3)
   Chlorogenic acid extract from the green beans of Coffea Arabica at a concentration ratio of 1:1:1. show synergistic anti-Hyaluronidase activity (IC50 μg/ml) of 6.7. While the extract of tender coconut water provides a continued cell repair and renewal mechanism, the seed extract from the members of the genus Nelumbo and the chlorogenic acid extract from Coffea arabica provide a protective mechanism to effectively tackle various forms of extrinsic skin damage.
6. Anti-skin damage compositions, said compositions comprising:
   1) extract of tender coconut water;
   2) seed extract from the members of the genus Nelumbo; and
   3) leaf extract of Murraya koenigii at a concentration ratio of 1:1:1.
   More specifically, the said composition comprises
   1) from about 100 mg to 1000 mg of tender coconut water extract;
   2) from about 100 mg to 1000 mg of seed extract from the members of the genus Nelumbo; and
   3) from about 100 mg to 1000 mg of leaf extract of Murraya koenigii.
   Still more specifically, 1) the extract of tender coconut water; 2) seed extract from the members of the genus Nelumbo; and 3) leaf extract of Murraya koenigii at a concentration ratio of 1:1:1. show synergistic anti-Hyaluronidase activity (IC50 μg/ml) of 4.3. While the extract of tender coconut water provides a continued cell repair and renewal mechanism, the seed extract from the members of the genus Nelumbo and the leaf extract from Murraya koenigii provide a protective mechanism to effectively tackle various forms of extrinsic skin damage.
7. Anti-skin damage compositions, said compositions comprising:
   1) extract of tender coconut water; and
   2) triterpene pentapeptide of Oleanolic acid at a concentration ratio of 7:3.
   More specifically, the composition comprises,
   1) from about 70 mg to 700 mg of tender coconut water extract;
   2) from about 30 mg to 300 mg of triterpene pentapeptide of Oleanolic acid.
   Further, 1) the extract of tender coconut water; and 2) triterpene pentapeptide of Oleanolic acid at a concentration ratio of 7:3 show synergistic anti-Hyaluronidase activity (IC₅₀ μg/ml) of 10. While the extract of tender coconut water provides a continued cell repair and renewal mechanism, the triterpene pentapeptide of Oleanolic acid provides a protective mechanism to effectively tackle various forms of extrinsic skin damage.
8. Anti-skin damage compositions, said compositions comprising:
   1) extract of tender coconut water; and
   2) triterpene pentapeptide of Ursolic acid at a concentration ratio of 7:3.
   More specifically the composition comprises,
   1) from about 70 mg to 700 mg of tender coconut water extract;
   2) from about 30 mg to 300 mg of triterpene pentapeptide of Ursolic acid.
   Further, 1) the extract of tender coconut water; and 2) triterpene pentapeptide of Ursolic acid at a concentration ratio of 7:3 show synergistic anti-Hyaluronidase activity (IC₅₀ μg/ml) of 12. While the extract of tender coconut water provides a continued cell repair and renewal mechanism, the triterpene pentapeptide of Ursolic acid provides a protective mechanism to effectively tackle various forms of extrinsic skin damage

In preferred embodiments the synergistic anti-hyaluronidase activities mentioned in the aforesaid paragraphs are presented in example I (Table I, FIG. I and FIG. II)

### Example I

TABLE I

| Serial | | | | |
|---|---|---|---|---|
| No | ANTI-HYALURONIDASE | | ACTIVITY (IC₅₀ μg/ml) | |
| 1. | Tender | Fruit ring | Tender coconut | |
| | coconut | extract of | water extract and | |
| | water | Garcinia | fruit ring extract of | |
| | extract | cambogia | Garcinia cambogia | |
| | | | at the concentration | |
| | | | ratio of 1:1 | |
| | 0 | 5 | 4.3 | |
| 2. | Tender | Fruit ring | Tender coconut | |
| | coconut | extract of | water extract and | |
| | water | Garcinia | fruit ring extract of | |
| | extract | mangostana | Garcinia mangostana | |
| | | | at the concentration | |
| | | | ratio of 1:1 | |
| | 0 | 2.5 | 7.6 | |
| 3. | Tender | Seed coat | Tender coconut | |
| | coconut | extract from | water extract and | |
| | water | Tamarindus | seed coat extract of | |
| | extract | indicus | Tamarindus indicus | |
| | | | at the concentration | |
| | | | ratio of 1:1 | |
| | 0 | 0.8 | 1.2 | |
| 4. | Tender | Seed coat | Seed extract from | Extract of tender |
| | coconut | extract of | the members of the | coconut water, the |
| | water | Tamarindus | genus Nelumbo | seed coat extract of |
| | extract | indicus | | Tamarindus indicus |
| | | | | and the seed |
| | | | | extract from the |
| | | | | members of the |
| | | | | genus Nelumbo at a |
| | | | | concentration ratio |
| | | | | of 1:1:1. |
| | 0 | 0.8 | 1.29 | 1.58 |
| 5. | Tender | Chlorogenic | Seed extract from | Extract of tender |
| | coconut | acid extract | the members of the | coconut water; |
| | water | of Coffea | genus Nelumbo | 2)seed extract from |
| | extract | arabica | | the members of the |
| | | | | genus Nelumbo; |
| | | | | and 3) Chlorogenic |
| | | | | acid extract of |
| | | | | Coffea arabica at a |
| | | | | concentration ratio |
| | | | | of 1:1:1. |
| | 0 | 8 | 1.29 | 6.7 |
| 6. | Tender | Leaf extract | Seed extract from | The extract of |
| | coconut | of Murraya | the members of the | tender coconut |
| | water | koenigii | genus Nelumbo | water; 2)seed |
| | extract | | | extract from the |
| | | | | members of the |
| | | | | genus Nelumbo; |
| | | | | and 3) leaf extract |
| | | | | of Murraya koenigii |
| | | | | at a concentration |
| | | | | ratio of 1:1:1 |
| | 0 | 37.5 | 1.29 | 4.3 |
| 7. | Tender | Triterpene | The extract of | |
| | coconut | pentapeptide | tender coconut | |
| | water | of Oleanolic | water; and 2) | |
| | extract | acid | triterpene | |
| | | | pentapeptide of | |
| | | | Oleanolic acid at a | |
| | | | concentration ratio | |
| | | | of 7:3 | |
| | 0 | 20 | 10 | |
| 8. | Tender | Triterpene | The extract of | |
| | coconut | pentapeptide | tender coconut | |
| | water | of Ursolic | water; and 2) | |
| | extract | acid | triterpene | |
| | | | pentapeptide of | |
| | | | Ursolic acid at a | |
| | | | concentration ratio | |
| | | | of 7:3 | |
| | 0 | 20 | 12 | |

The invention presents the following advantageous features.
a) The present invention provides skin care compositions containing natural plant extracts, wherein the compositions exhibit a complimentary dual mode of action by (a) ensuring the viability and constant renewal of the cells of the skin; (b) ensuring skin protection by virtue of “skin care” properties of other natural actives incorporated in the composition.
b) The present invention provides skin care compositions containing natural plant extracts, wherein each of the natural extracts have been carefully chosen based on useful cosmetic properties.
c) The present invention provides skin care compositions containing natural plant extracts that exhibits an enhanced safety profile for application on the skin.
d) The present invention provides skin care compositions containing natural plant extracts that exhibits an enhanced ability to counter the problems of both intrinsic and extrinsic skin insults, through a complimentary effect.
e) The present invention provides skin care compositions containing natural plant extracts wherein the compositions are suited to address the multitude of problems associated with skin insult including but not limited to thinning of the skin, reduction in the number of skin cells, reduction in the number of blood vessels that supply the skin and hence reduction in the microcirculation of the skin, flattening of the dermal-epidermal junction which may cause weaker mechanical resistance of the skin, appearance of fine lines, wrinkles (both the superficial wrinkles and deep/coarse wrinkles), crevices, bumps, hyper-pigmentation in the forms of age spots and freckles, sallowness, sagging, loss of skin firmness, loss of skin elasticity, skin blotching, dark under-eye circles, puffy eyes, enlarged skin pores, diminished rate of cell turnover, abnormal desquamation or exfoliation or the skin cells and visible dead skin caused due to flaking and scaling.
f) The present invention provides skin care compositions containing natural plant extracts wherein the compositions are suited to address specific problems or combination of one or more problems or the multitude of problems associated with skin insult through a combination of various mechanisms including not limited to rejuvenation of skin by providing nutrition, improving the cell turn over, improving skin microcirculation, stimulating collagen and elastin fiber synthesis, inhibition of serine proteases such as collagenases and elastases from acting on collagen and elastin in the dermis of the skin, maintenance of the “acid mantle” produced by the sebaceous and sweat glands to confer a healthy glow to the skin, relaxation of the skin, soothening and softening dry skin and providing powerful anti-oxidant systems that will effectively counter the deleterious effects of free radicals like nitric oxide, superoxide radicals, hydrogen peroxide, and hydroxide free radicals generated by oxidative processes.
g) The present invention provides skin care compositions containing natural plant extracts, wherein the said composition exhibits enhanced stability by virtue of the stability evinced by the individual plant extracts included therein.
h) The present invention provides skin care compositions containing natural plant extracts which can be combined with one or more cosmetic or manufacturing adjuncts including but not limited to sunscreen agents, skin-lightening agents, skin-tanning agents, other antioxidants, perfumes, opacifiers, preservatives, colorants, emulsifiers, thickeners, buffers and the like.
i) The present invention provides skin care compositions containing natural plant extracts which can be administered by the topical mode or oral mode or co-administered by both the topical and oral modes.
j) The present invention provides a natural, comprehensive, multi-component, multi-functional, safe and stable prophylactic and therapeutic solution for the entire gamut of pathological changes associated with skin insults.
k) The present invention also provides a natural, comprehensive, multi-component, multi-functional, safe and stable prophylactic and therapeutic solution that would help individuals manage the psychosocial aspects of skin insults, including but not limited to social anxiety, social isolation, poor self image, eating disorders, body dysmorphic disorder, chronic illnesses, workplace discrimination, lack of confidence and depression.

While the invention has been described with reference to a preferred embodiment, it is to be clearly understood by those skilled in the art that the invention is not limited thereto. Rather, the scope of the invention is to be interpreted only in conjunction with the appended claims.

We claim:

1. Anti-skin damage compositions comprising the extract of tender coconut water and the fruit ring extract of Garcinia cambogia in a concentration ratio of 1:1, wherein said composition protects the skin from insult through a dual complementary mode of action involving (i) continued cell repair and renewal mechanism; and (ii) protection against various forms of extrinsic skin damage

2. The compositions according to claim 1, wherein the extract of tender coconut water and the fruit ring extract of Garcinia cambogia at a concentration ratio of 1:1 synergistically shows a hyaluronidase activity of IC₅₀ at 4.3 μg/ml.

3. Anti-skin damage compositions, comprising the extract of tender coconut water and the fruit ring extract of Garcinia mangostana at a concentration ratio of 1:1, wherein said composition protect the skin from insult through a dual complementary mode of action involving (i) continued cell repair and renewal mechanism; and (ii) protection against various forms of extrinsic skin damage

4. The compositions according to claim 3, wherein the extract of tender coconut water and the fruit ring extract of Garcinia mangostana at a concentration ratio of 1:1 synergistically shows a hyaluronidase activity of IC₅₀ at 7.6 μg/ml.

5. Anti-skin damage compositions, comprising the extract of tender coconut water and the seed coat extract of Tamarindus indicus at a concentration ratio of 7:3, wherein said composition protects the skin from insult through a dual complementary mode of action involving (i) continued cell repair and renewal mechanism; and (ii) protection against various forms of extrinsic skin damage.

6. The composition according to claim 5, wherein the extract of tender coconut water and the seed coat extract of Tamarindus indica at a concentration ratio of 7:3 show synergistic anti-hyaluronidase activity with an IC50 (μg/ml) of 1.2.

7. Anti-skin damage compositions comprising the extract of tender coconut water, the seed coat extract of Tamarindus indicus and the seed extract from the members of the genus Nelumbo at a concentration ratio of 1:1:1, wherein said composition protects the skin from insult through a dual complementary mode of action involving (i) continued cell repair and renewal mechanism; and (ii) protection against various forms of extrinsic skin damage.

8. The compositions according to claim 7, wherein the extract of tender coconut water, the seed coat extract of Tamarindus indicus and the seed extract from the members of the genus Nelumbo at a concentration ratio of 1:1:1 shows synergistic anti-hyaluronidase activity with an IC50 (μg/ml) of 1.58.

9. Anti-skin damage compositions comprising
1) extract of tender coconut water;
2) seed extract from the members of the genus Nelumbo; and
3) Chlorogenic acid extract from the green beans of Coffea Arabica
at a concentration ratio of 1:1:1, wherein said composition protects the skin from insult through a dual complementary mode of action involving (i) continued cell repair and renewal mechanism; and (ii) protection against various forms of extrinsic skin damage.

10. The composition according to claim 9, wherein 1) the extract of tender coconut water; 2) seed extract from the members of the genus Nelumbo; and 3) Chlorogenic acid extract from the green beans of Coffea Arabica at a concentration ratio of 1:1:1. show synergistic anti-Hyaluronidase activity (IC50 μg/ml) of 6.7.

11. Anti-skin damage compositions, said compositions comprising:
1) extract of tender coconut water;
2) seed extract from the members of the genus Nelumbo; and
3) leaf extract of Murraya koenigii at a concentration ratio of 1:1:1, wherein said composition protects the skin from insult through a dual complementary mode of action involving (i) continued cell repair and renewal mechanism; and (ii) protection against various forms of extrinsic skin damage.

12. The composition according to claim 11, wherein 1) the extract of tender coconut water; 2) seed extract from the members of the genus Nelumbo; and 3) leaf extract of Murraya koenigii at a concentration ratio of 1:1:1. show synergistic anti-Hyaluronidase activity (IC50 μg/ml) of 4.3.

13. Anti-skin damage compositions, said compositions comprising:
1) extract of tender coconut water; and
2) triterpene pentapeptide of Oleanolic acid at a concentration ratio of 7:3, wherein said composition protects the skin from insult through a dual complementary mode of action involving (i) continued cell repair and renewal mechanism; and (ii) protection against various forms of extrinsic skin damage.

14. The composition according to claim 13, wherein 1) the extract of tender coconut water; and 2) triterpene pentapeptide of Oleanolic acid at a concentration ratio of 7:3 show synergistic anti-Hyaluronidase activity (IC50 μg/ml) of 10.

15. Anti-skin damage compositions, said compositions comprising:
1) extract of tender coconut water; and
2) triterpene pentapeptide of Ursolic acid at a concentration ratio of 7:3, wherein said composition protects the skin from insult through a dual complementary mode of action involving (i) continued cell repair and renewal mechanism; and (ii) protection against various forms of extrinsic skin damage.

16. The composition according to claim 15, wherein 1) the extract of tender coconut water; and 2) triterpene pentapeptide of Ursolic acid at a concentration ratio of 7:3 show synergistic anti-Hyaluronidase activity (IC50 μg/ml) of 12.
